(19) United States
(12) Patent Application Publication
(10) Pub. No.: US 2006/0135844 A1
(43) Pub. Date: Jun. 22, 2006

(54) Therapeutic device for local area stimulation

(76) Inventor: Nikolay Alekseyenko, Pacifica, CA (US)

Correspondence Address: Nikolay Alekseyenko, 154 Outlook Circle, Pacifica, CA, 94044, US

(21) Appl. No.: 11/002,781

(22) Filed: Dec. 2, 2004

(51) Int. Cl. A61N 2/00; A61B 17/52

(52) U.S. Cl. 600/9

(57) ABSTRACT

A therapeutic device comprised of an arrangement of points in an array (100, 112, 130, 172, 174) in flexible mounts (102, 112, 120, 122, 140, 144, 146, 182). The points are connected by electrically conductive materials (106, 108) permitting application of electromagnetic forces in easily varied configurations of accessories (178, 80) with options for monitoring and control (160, 162, 168, 170). The preferred embodiment (172, 176) is designed for simplicity, convenience and portability. An alternative embodiment (174) accommodates additional sensors and therapeutic devices suitable for clinical use and scientific research.

### BACKGROUND

1. Description of Prior Art

Medical practitioners in a wide variety of disciplines and traditions have long observed the value of various local stimuli in therapy.

From hot water bottles to acupuncture needles, from electrical currents to magnetic fields, and from massages to plasters, many stimuli are recognized as helpful components in therapy. They are administered continuously or intermittently, constant or modulated, over a wide range of intensities from extremely fine to much higher, and, in the case of electrical and magnetic stimuli, with constant or alternating polarities.

Fields of practice using such stimuli include sports and rehabilitative medicine, physical therapy, acupuncture, acupressure, therapeutic massage, and less traditional practices such as magneto-therapy and reflexive therapy.

A number of devices have been invented to aid in administering therapeutic stimuli.

One device for intramuscular stimulation therapy applies local electric currents is proposed in U.S. Pat. No. 6,058,938 to Chu and Styles (1998). This device has an electric battery power source connected to conductive tips held on Teflon handles and inserted subcutaneously.

Shortcomings of this device include:
a. It is necessarily invasive, requiring insertion of the points.
b. It lacks control over the polarity and amount of current applied.
c. The device itself is not responsive to changes in the patient, such as polarization and resistivity around the inserted electrodes, during the course of administration.
d. It is dependent upon external power supplies.
e. It does not administer other stimuli such as heat and precludes their simultaneous application.

Another related device is proposed in U.S. Pat. No. 4,590,939 to Sakowski (1984). It is used to reduce pain according to acupressure principles through application of local mechanical pressure.

Shortcomings of this device are that its use precludes simultaneous administration of other stimuli singly or in combination. The gross mechanical motions it induces are incompatible with numerous conditions including lesions, surgical wounds, bruising, ulcerations, or infections.

A third device is proposed in U.S. Pat. No. 4,319,574 to Sun and Sun (1980). It also applies physical pressure and is used to stimulate “Biologically Active Spots” (BAS). It consists of a flat plate with two protruding parts that move against each other by means of an electromagnet. The parts have adjustable positions set by selectable stops.

Shortcomings of this device include:
a. It neither provides nor permits simultaneous application of other stimuli singly or in combination.
b. It requires an external electrical power supply.
c. It requires time-consuming procedures to set up.
d. It is dependent upon external power supplies.
e. It does not administer other stimuli such as heat and precludes their simultaneous application.

These devices are limited to administration of one type of stimulus and make simultaneous administration of other stimuli impractical. They are very localized to one point, or, in the case of the first device, a line between two points.

2. Objects and Advantages of the Present Invention

Accordingly, besides the objects and advantages of the device described in the above description of prior art, several objects and advantages of the present invention are:
a. to provide a gentle and non-invasive physical stimulus across an area of a subject's body surface;
b. to provide fine degrees of electrical potential or current or magnetic fields without need of external power supplies;
c. to apply stimuli evenly over the area of application;
d. to optionally apply stimuli in differing degrees or kinds to selectable sub-portions of the area of application;
e. to optionally apply various types of electrical, modulated, or magnetic stimuli as required;
f. to optionally apply heat to the area of application;
g. to monitor physical changes, for example field polarities, magnetic coercivity, or electrical resistance at selectable areas of application during a course of treatment;
h. to provide electrical and magnetic stimuli in any strength needed;
i. to provide variation, modulation, and control of electromagnetic stimuli over time;
j. to accommodate simultaneous topical or subcutaneous administration of medicinal substances and/or additional, highly localized stimuli;
k. to activate, potentiate, or synergistically enhance medicinal substances by means of electrical, magnetic and/or thermal fields;
l. to optionally function autonomously without external power sources or with external power sources as appropriate;
m. to provide optional self-regulating or manual regulation of any therapeutic stimuli;
n. to provide a variety of immediately available configurations, portability, and minimal setup;
o. to provide the above functionality in separable components or modules which operate singly or in combination.
p. to provide optional means for additional therapeutic administrations including, but not limited to, invasive techniques, additional controls, or monitoring devices.
q. to provide, through replaceable modules, a wide variety of sources of stimuli, of controllers—both manual and automated—for said stimuli, and means of monitoring the stimuli, their effects, and pertinent conditions or responses within this instrument's domain of influence.
r. to provide a platform for quantifiable, scientific research into extended ranges and combinations of hitherto mutually exclusive therapies. By using the unprecedented variety field effects this instrument, with supporting instruments, can handle, it may lead, we should hope, to deeper understandings of health and healing.

### SUMMARY

In accordance with the present invention the therapeutic device for applying stimuli is an array of metal points arranged in rows alternating between ferromagnetic and diamagnetic materials, and held in a flexible, magnetized support so connected and arranged as to administer a wide variety of physical stimuli singly or in combination.

### DRAWINGS

Drawing Figures

In the drawings, closely related figures have the same number but different alphabetic suffixes.

FIG. 1A is an array of points.

FIG. 1B is a flexible base block.

FIG. 1C is a point array embedded in a flexible base block, viewed from the active [front] side.

FIG. 2A is a point row.

FIG. 2B is a conductive strip.

FIG. 2C is the point row and the conductive strip assembled into a point row assembly.

FIG. 2D is a distal [rear] view of an entire point row assembly.

FIG. 2E is a close-up of a point row assembly.

FIG. 3A is an array of points with alternating rows of two materials, ferromagnetic and diamagnetic metals.

FIG. 3B is a flexible base block with mounting points.

FIGS. 3C and 3D are point row assemblies in the flexible base block-active side view and side elevation, respectively.

FIG. 3E is a unit point block assembly, exploded view, of the preferred [portable] embodiment.

FIGS. 4A and 4B show unit point block assemblies in distal [away from patient] side views highlighting the alternating point materials and the conductive strips, respectively, in the preferred [portable] embodiment

FIG. 5A shows a 3×3 assembly of unit blocks, an arbitrary array of separably controllable unit point block assemblies—in three rows of three in this example, in the preferred [portable] embodiment.

FIG. 5B shows a 3×3 unit block assembly attached to a flexible base (e.g. a sturdy fabric) in the preferred embodiment.

FIG. 6A is a 3×3 unit assembly on its base with electrical connectors to two source modules supplying electromagnetic effects through an intermediary adapter module in the preferred embodiment.

FIG. 6B is an example of a finished operational assembly showing accessory attachment points in its flexible base in the preferred [portable] embodiment.

FIGS. 7A and 7B shows an operational assembly, alternative [clinical] embodiment, with two attachments in an exploded view and assembled, respectively. The visco-elastic pad attachment is usually employed. Other accessories, optional, fit between the operational assembly and its visco-elastic pad attachment.

FIG. 7C is an exploded view of a typical attachable accessory, in this case providing supplementary magnetic fields by means of permanent magnets. Access holes and gaps within the accessory assembly make it fully compatible with the preferred [portable] and the alternative [clinical] embodiments of this invention.

FIGS. 8A and 8B show plan views of an electromechanical adapter with one and two source modules, respectively, attached to the preferred embodiment of this invention.

FIG. 8C is a standard electromechanical 3×3 adapter module with a source module.

FIG. 8D is a 3×3 data and real time adapter module with a data source module, permitting individually addressable monitoring and control of each point block.

FIG. 9A shows the alternative [clinical] embodiment support block. The opening in the flexible base accommodates a mounting fixture for accessories including hypodermic needles, topical medicinal applicators, and sensors to monitor physical parameters such as temperatures, or electromagnetic and galvanic, conditions.

FIG. 9B and FIG. 9C show an alternative embodiment unit point block assembly in active and distal side views, respectively.

FIG. 9D is a unit point block assembly of the alternative [clinical] embodiment in an exploded view.

### DRAWINGS—REFERENCE NUMERALS

100: Point Array
102: Simple Support Block
104: Row of Points
106: Conductive Strip
108: Conductive Strip Wire Lead
110: Point Row Assembly
111: Point Row Assembly [close-up]
112: Points Assembled in Block
114: Ferromagnetic Metal Point Rows
116: Diamagnetic Metal Point Rows
120: Support Block, Preferred Embodiment
122: Support Block Mounting Points
124: Support Block, Alternative Embodiment
126: Opening for Clinical Accessory Mount
128: Clinical Accessory Mount
130: Unit Point Block Assembly, Preferred Embodiment
132: Unit Point Block Assembly, Alternative Embodiment
138: Fasteners to Assemble Unit Blocks
140: 3×3 Unit Assembly, Preferred Embodiment
142: 3×3 Unit Assembly, Alternative Embodiment
144: Fasteners
146: Flexible Base
148: Accessory Attachment Points, Grommeted-Style
150: Clinical Accessory Mount Access Openings, Grommeted
152: Grommets
160: Adapter Module, Electro-Mechanical
162: Adapter Module, Data and Real Time
164: Electrical Connector Adapter to Source Module
166: Electrical Connector Adapter to Unit Assembly
168: Source Module, Parallel
170: Source Module, Addressable Control
172: Operational Assembly. Dual Source, Preferred Embodiment
174: Operational Assembly, Dual Source, Alternative Embodiment
176: Operational Assembly, Single Source, Preferred Embodiment
178: Visco-Elastic Pad Attachment
180: Attachable Accessory
182: Flexible Support
184: Permanent Magnet Array
186: Gaps in Array Accommodate Attachment Access Lines
208: Source Selector Switch [Single-Pole, Triple-Throw]
210: Source On-Off-Option Switch [9-or-Greater Pole, Multi-Throw]
212: Function Selector Switch [18-or-Greater-Pole, Multi-Throw]
214: Multi-Circuit Jack for Source Module Connections
218: Attachment Points to Flexible Base
220: Multi-Circuit Connector Plug to Unit Blocks
222: Metering and Status Display
224: Variable Control
226: Input/Output Jacks
228: Multi-Circuit Analog Pass-Through Jack

### DETAILED DESCRIPTION OF THE INVENTION, PREFERRED EMBODIMENT

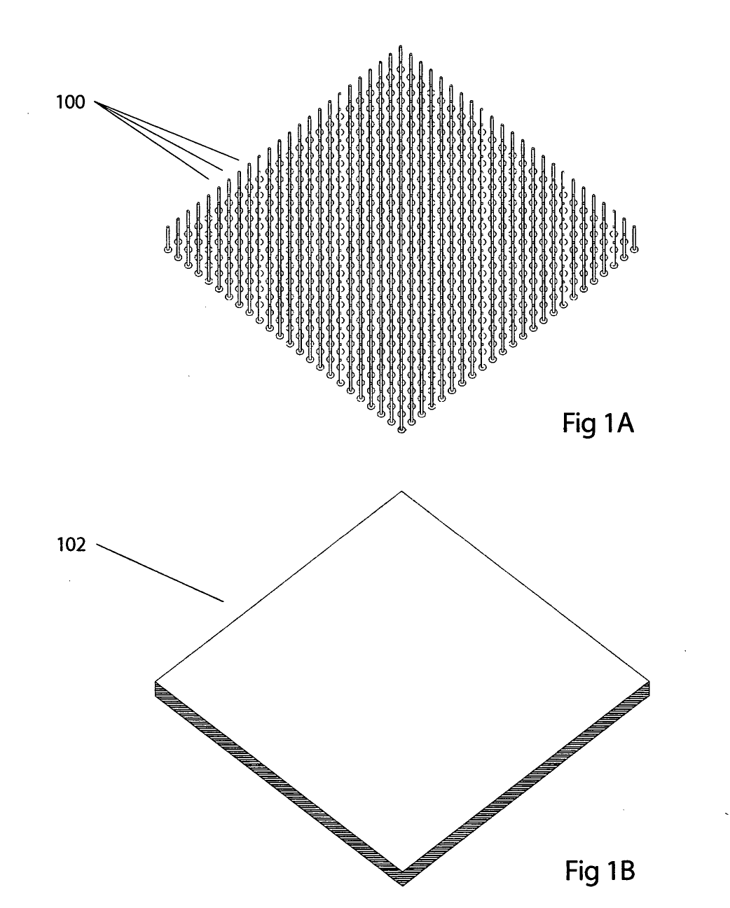

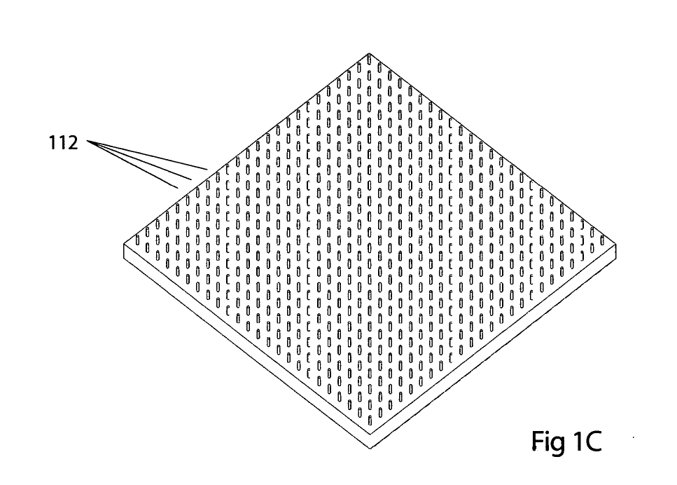

The novel and non-obvious essence of this invention is an array of points (FIG. 1A) and a support (FIG. 1B) assembled together (FIG. 1C). This point array provides a physical stimulus to the skin of the subject under treatment. It has been observed that this novel arrangement is unexpectedly able to induce healing effects beyond other forms of stimulation that attempt to therapeutically induce and support healing.

By using a flexible support in a planar array, it was found that the points could be quite sharp and not break the subject's skin because human skin is stretchy and the point arrays, flexing under the pressure of the subject lying on them, distribute the pressure evenly.

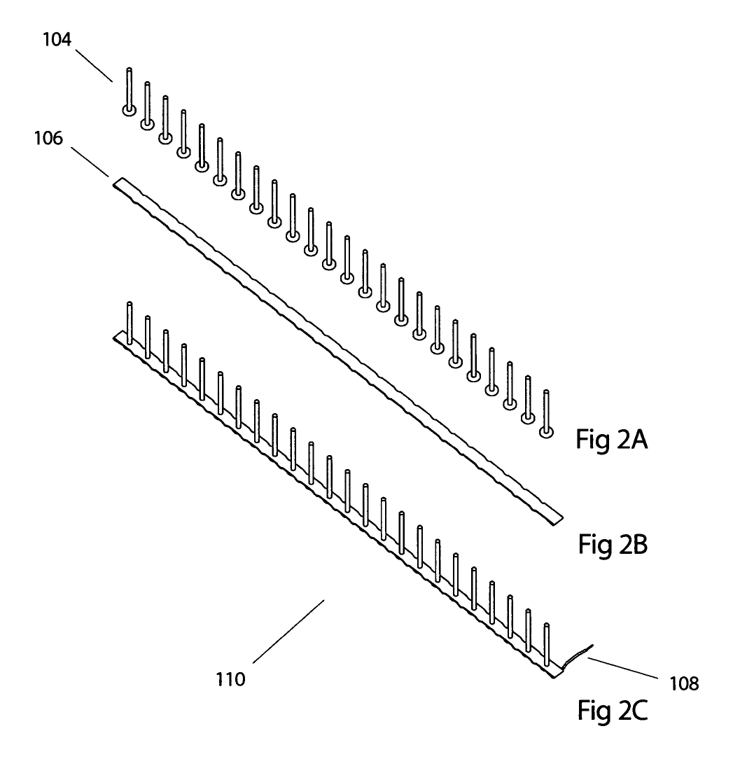

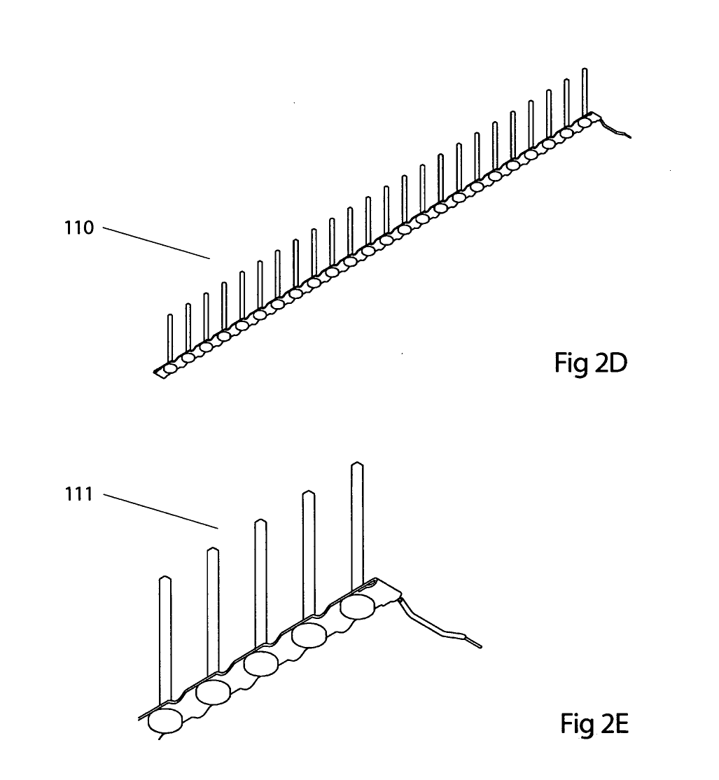

To this effect was added the use of metal points connected (FIG. 2A-2E) to permit application of electrical current or potential which enhanced the benefits in an unexpectedly strong fashion.

The flexible support is a synthetic, rubbery “magnetophore” material which is permanently magnetized by added ferromagnetic ingredients, similar to “refrigerator magnets.” This provides benefits of a magnetic field as a therapeutic stimulus.

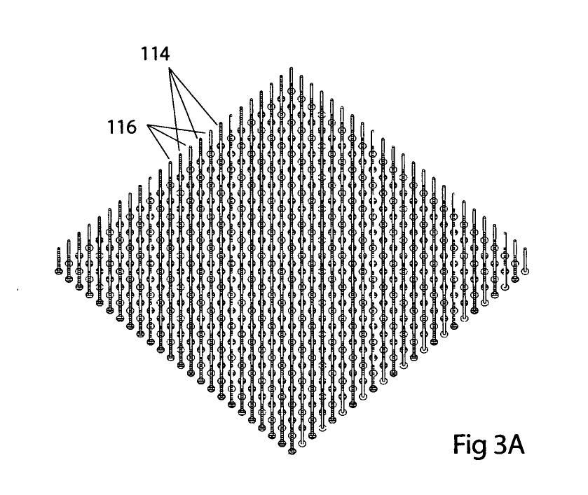

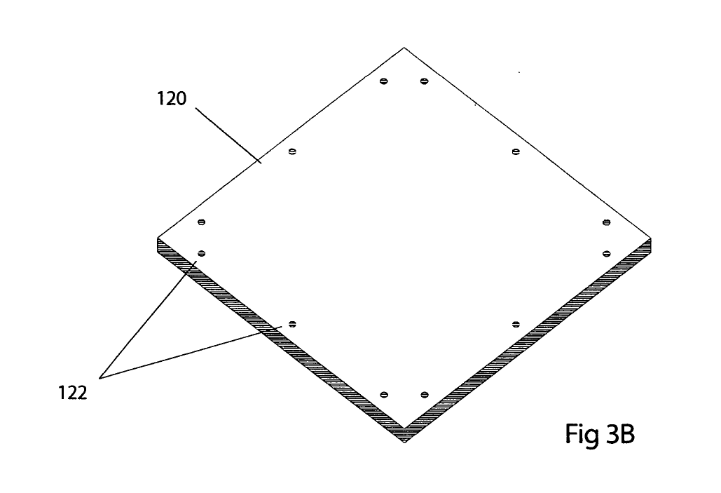

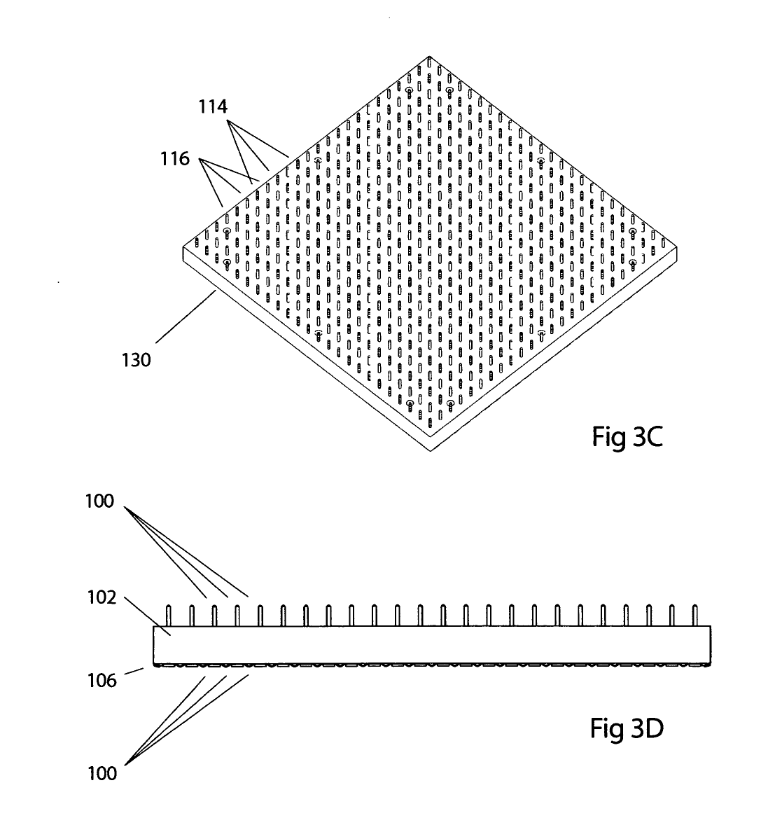

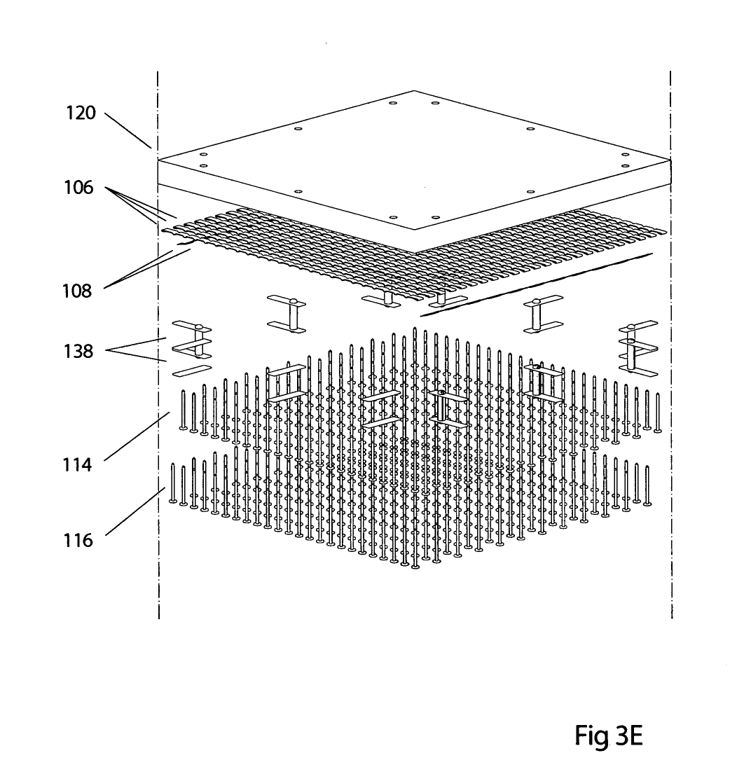

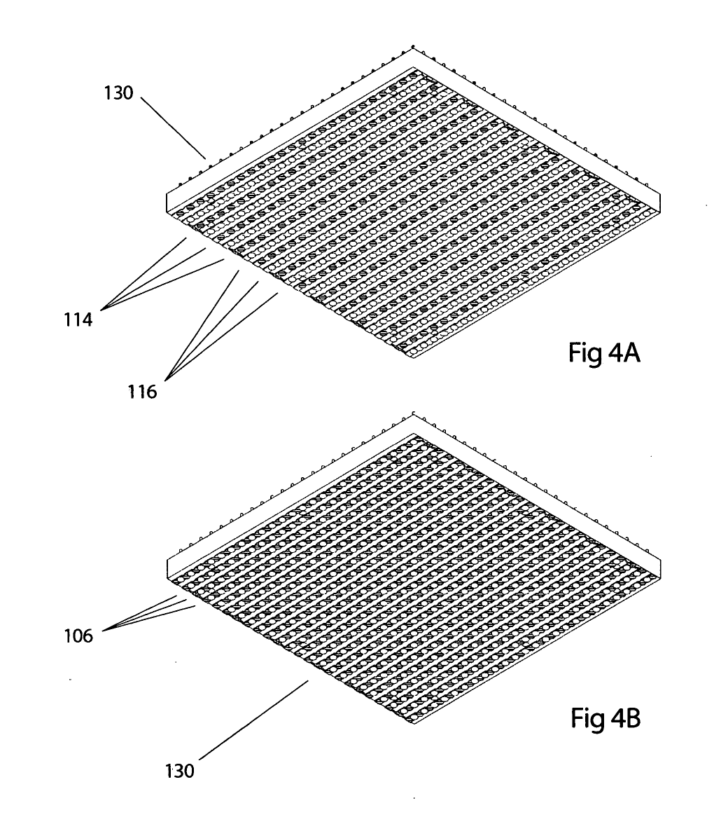

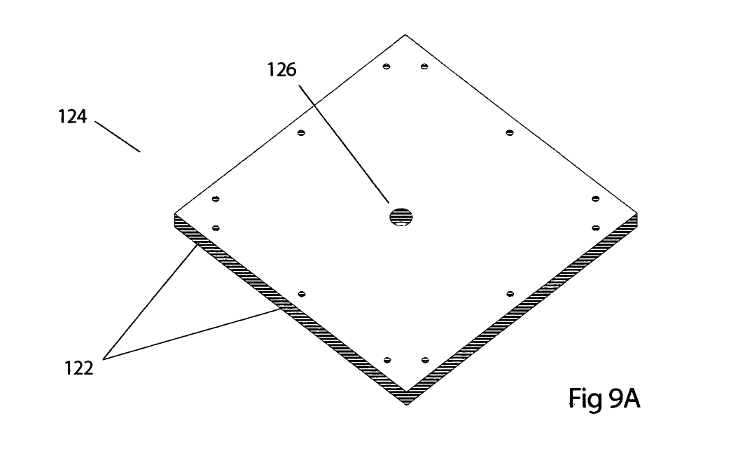

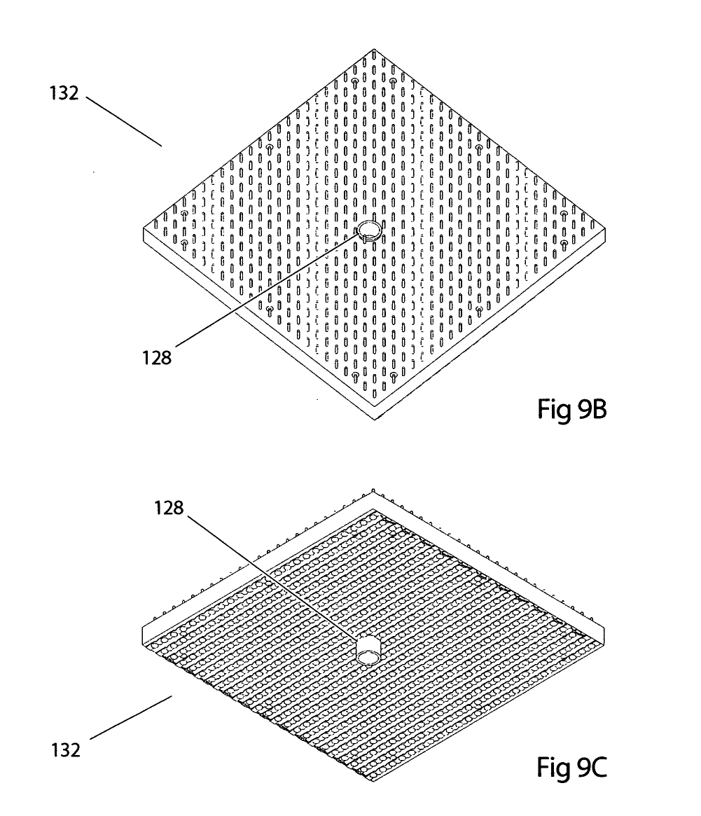

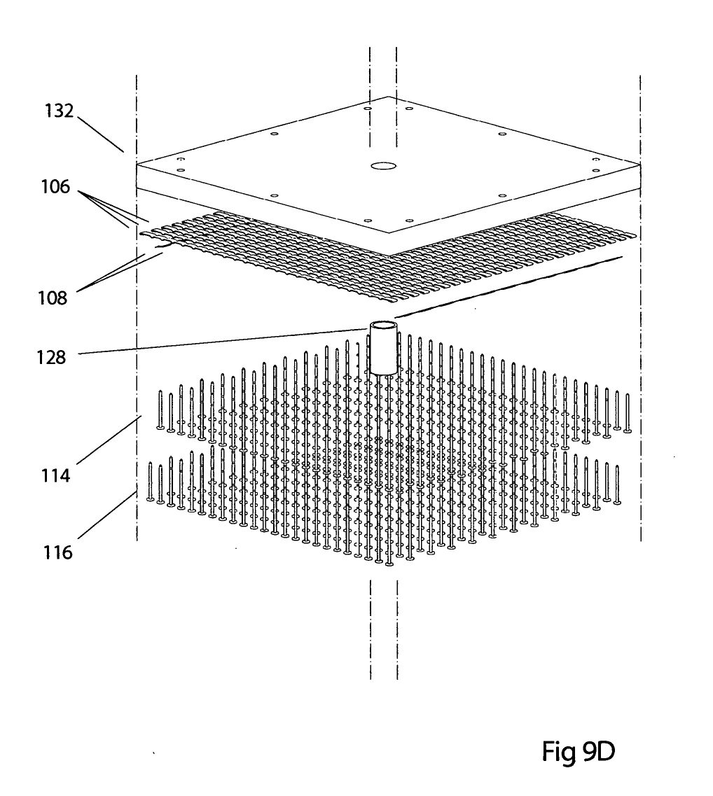

Further developments incorporated the galvanic response of a subject's skin to adjacent ferromagnetic and diamagnetic metal points (FIG. 3A, FIG. 3C, FIG. 3E, FIG. 4A-4B, FIG. 9C) which create micro voltages and currents when connected electrically due to the moist, saline, conductive character of human skin. This phenomenon yielded unexpected benefits differing from those derived from direct stimulation by applying electric current or fields.

A further unexpected and novel effect of the mixed ferromagnetic and diamagnetic points was seen in the sharp enhancement of the magnetic field arising from the magnetophore base which varies in relative polarity and intensity as it passes from point to point.

One arrangement of diamagnetic (e.g., brass or copper) and ferromagnetic (e.g., iron or steel) points, the arrangement used in the preferred embodiment of this invention, is in alternating rows, connected within themselves by a conductor and the rows connected with each other by wire leads (FIG. 2D-2E).

This novel device marks a significant advance over other electrical and magnetic stimulation devices in that the intermixed arrays of points create an effect of broad electrodes and, at the same time, multiple simultaneous point electrodes. The unexpected effect is to dramatically enhance the effectiveness of treatments.

The present state of research indicates that a frequency of 4 to 10 points per square centimeter is effective. Rectangular arrangements of points are practical to assemble but further research may indicate advantages for hexagonal or other distributions not shown in the present embodiments.

Current research indicates that the points should be sharp to be most effective. In practice, there are no attendant problems observed with points puncturing, breaking or irritating the subject's skin.

A further advance incorporated magnetized material into the point block supports (FIGS. 1B, 3B, 3E, 9A, 9D). The combination of an array of points with a broad magnetic field potentiates the magnetic forces much as electrical fields are seen to be enhanced. On one hand, the point arrays create a wide field and on the other, they create multiple instances of field changes from point to point.

This fundamental advance is seen in our preferred embodiment as an assembly referred to as the unit point block assembly (FIG. 3A to 3C, 4A to 4C)

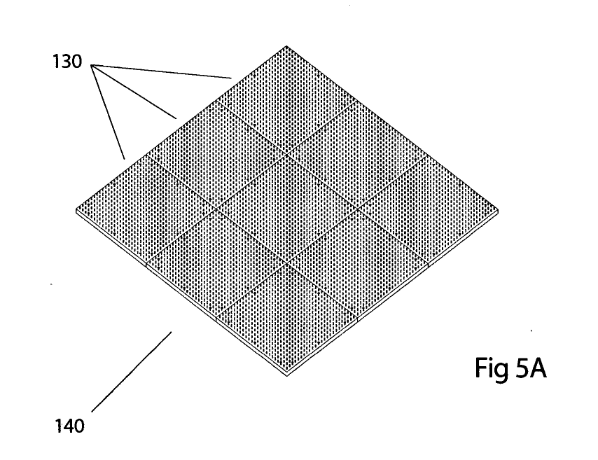

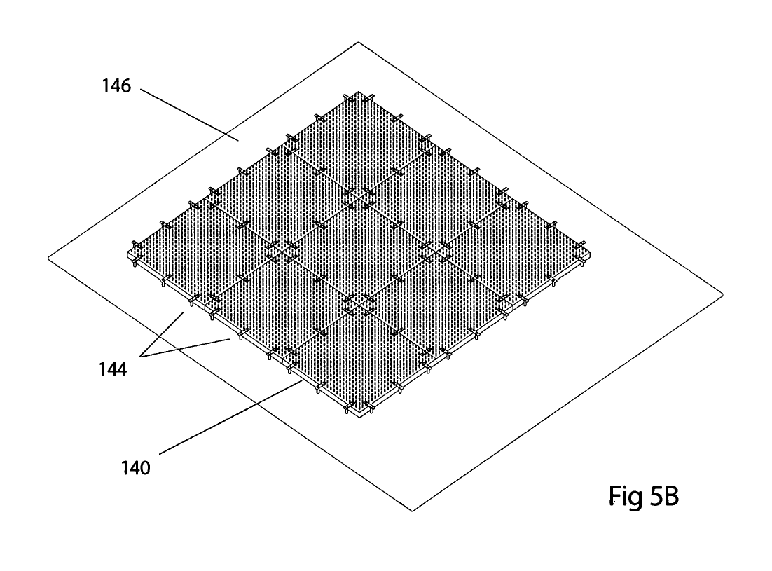

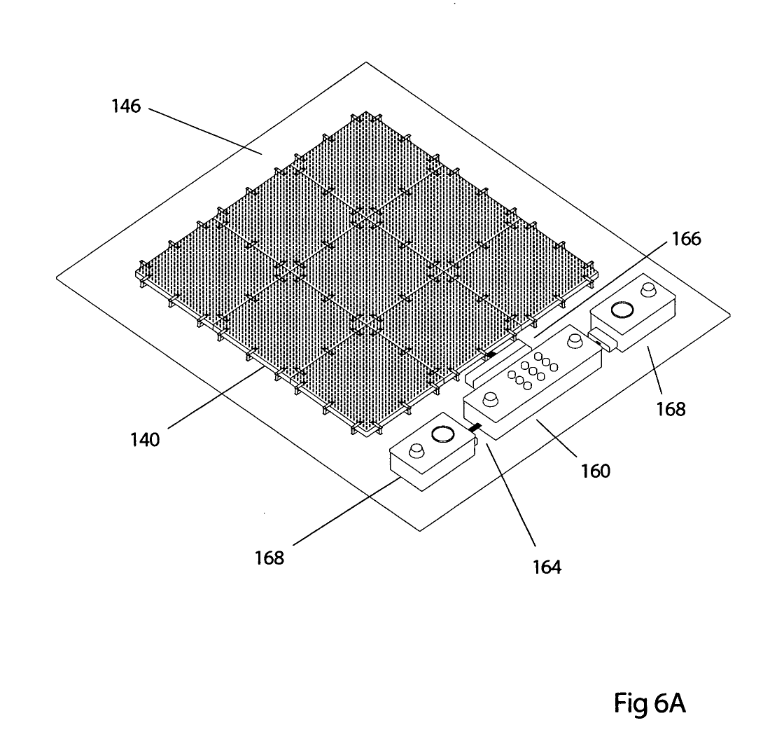

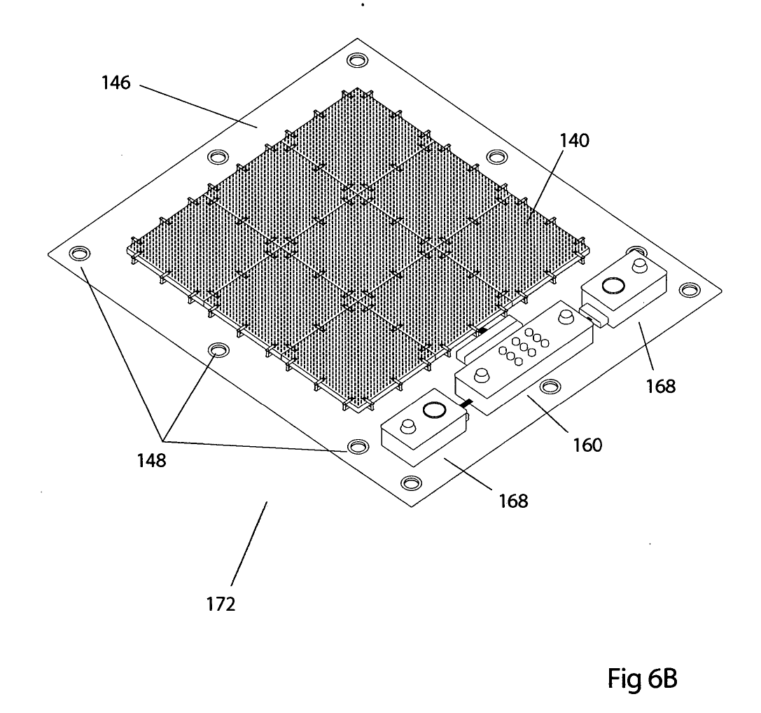

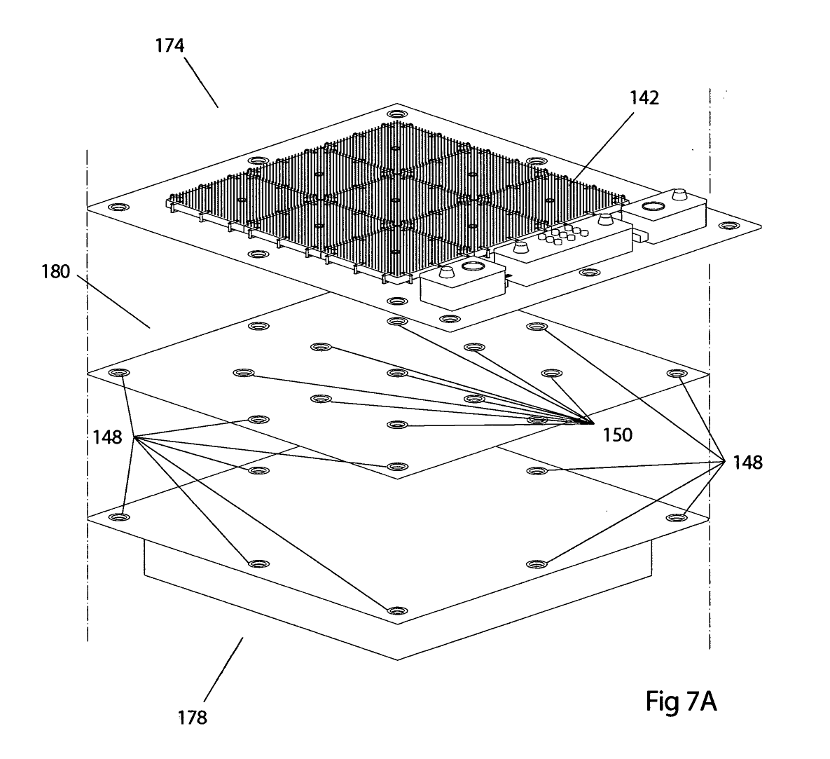

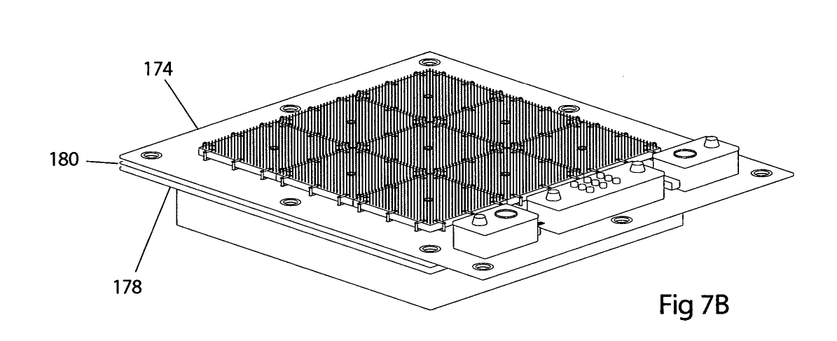

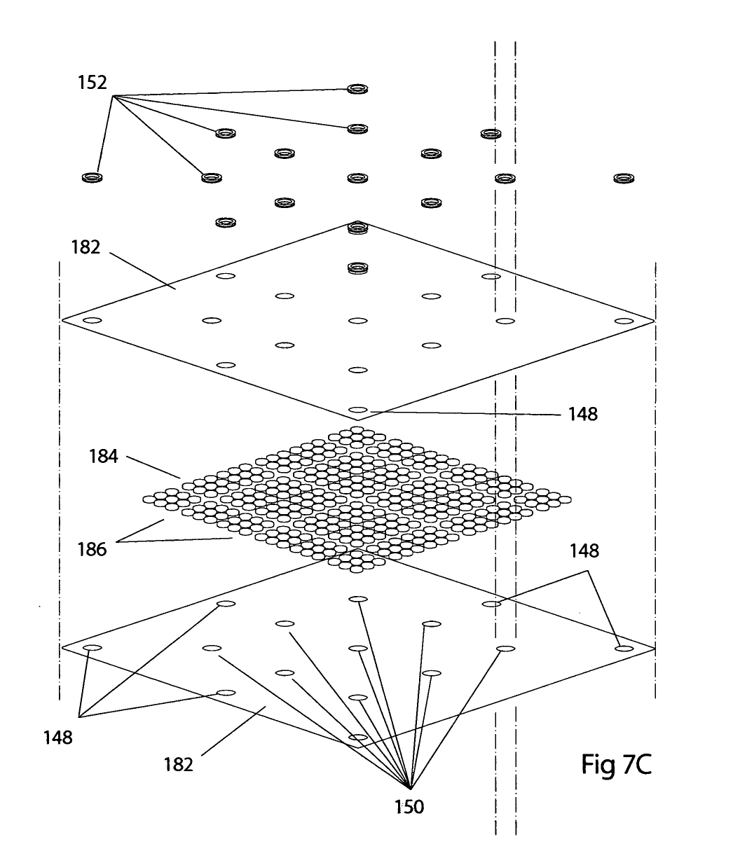

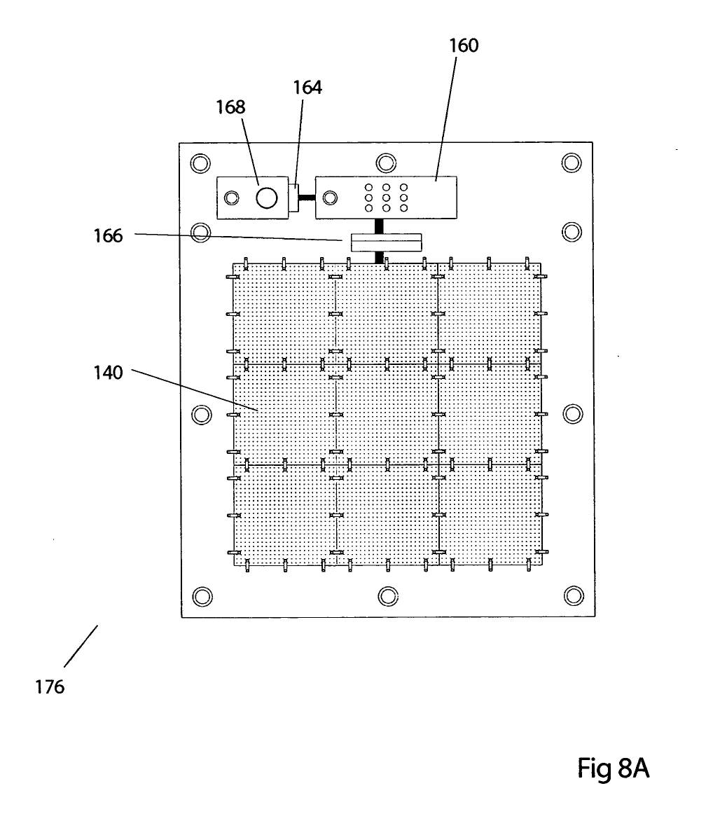

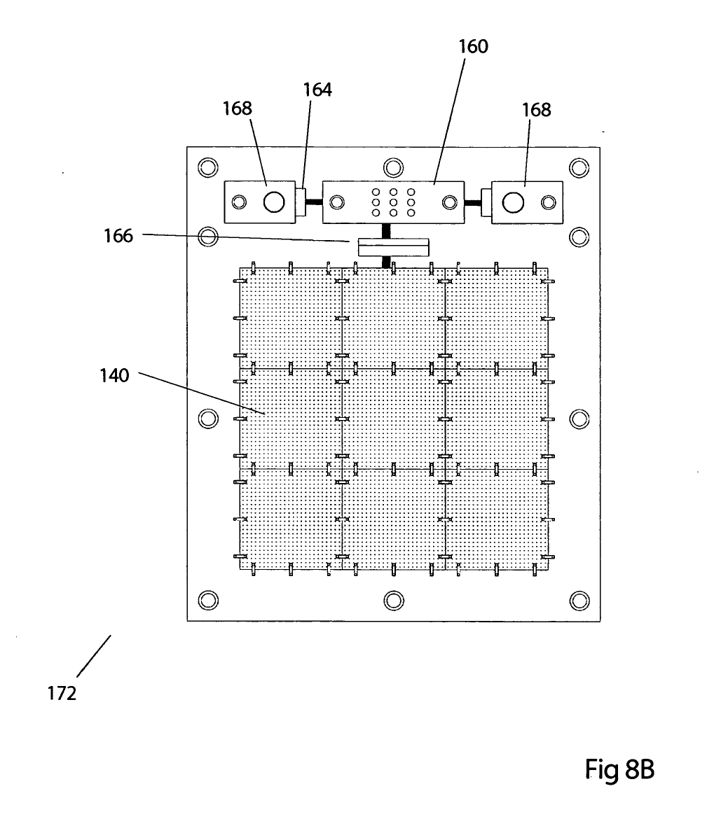

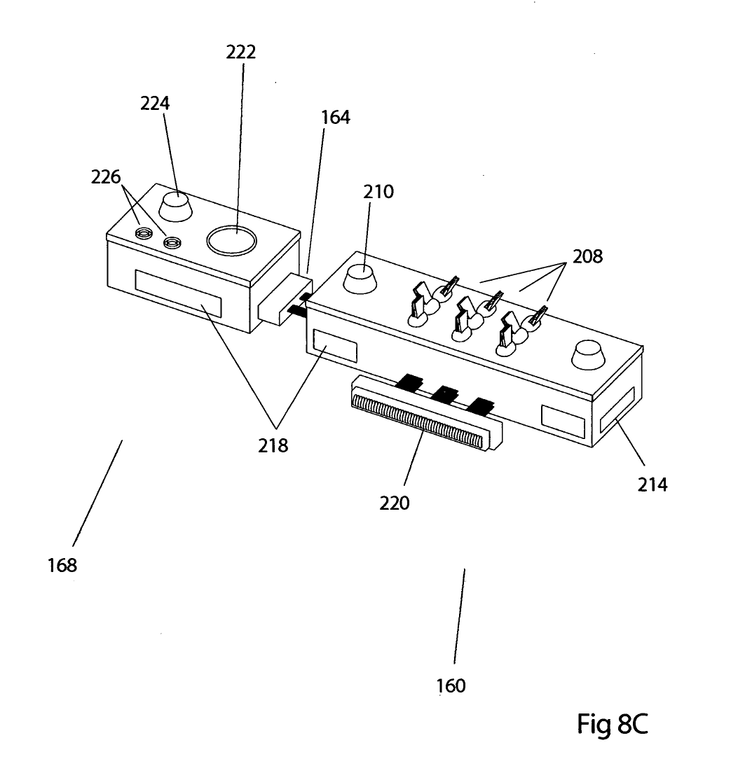

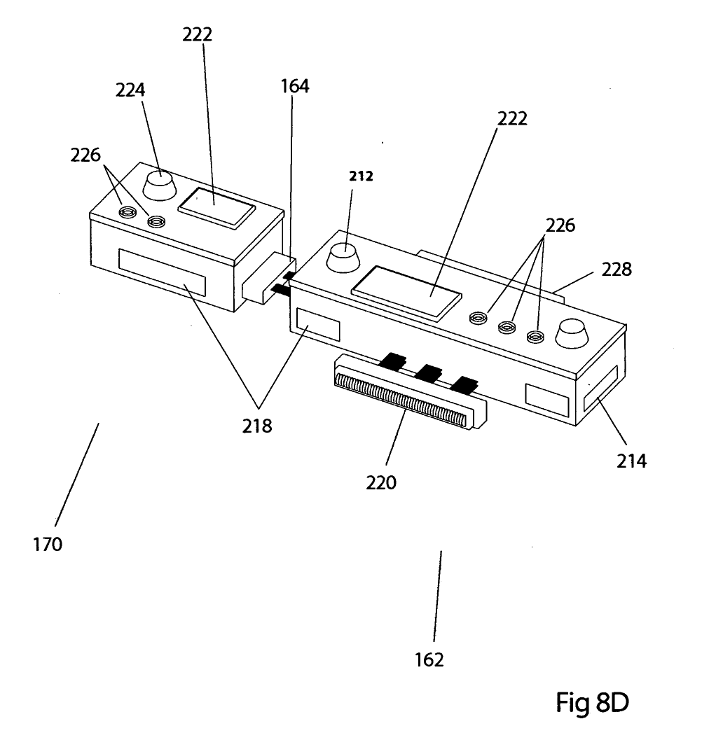

Assembling these unit blocks into arbitrarily larger arrays is easily accomplished with fasteners that allow flexibility across the whole device (FIG. 5B). Assemblies incorporate these larger arrays with a flexible base, wiring, connectors, and attachment points (FIG. 6A-6B) for power supplies and other component layers (FIG. 8A-8D. The other components such as heating pads, and enhanced magnetic force can be used as needed (FIG. 7A to 7C).

Description—Operational Modules

Therapeutic devices often apply up to 10 volts DC to a subject. The discovery of the potentiated value of micro currents, galvanically induced, presents the other extreme of electrical intensity useful in this device, a very wide range. Many different forms of electrical current are known to be useful or possibly useful.

Accordingly, it is important to allow the optional use of differing source modules (FIGS. 8A-D) or none at all. The adapter module proposed in both embodiments of this invention attach to any source modules (FIG. 8A-8D) and to the built-in wiring harness of the point block arrays, permitting use of source modules or local galvanic effects in a variety of combinations.

Description—Adapter Modules

The elecro-mechanical adapter module (6A, 6b, 8A-C, 160) is electromechanical in operation and permits individual switching of each point block assembly in the array between up to two source modules, its own galvanic conditions, or an open circuit (no current flow). Unit blocks connected to a source module function in parallel, including galvanically- and environmentally-induced loads. Blocks switched to “self” (the middle throw of the three position switch), each has its own separate galvanically- and environmentally-induced current.

A data and real time adapter module (FIG. 8D, 162) permits external devices to simultaneously monitor, drive or control each point block assembly. This direct or indirect control employs one or multiple circuits by analog signals through the multi-circuit analog jack (228) or via integrated digital controllers through input/output jacks (226). It employs commercially-available integrated circuits, controller boards, and operating systems. The arbitrary number of circuits available to each point block is limited only by practical considerations.

Description—Source Modules

Source modules (168, 170) can be self-contained, battery-powered, piezo-mechanically-powered, or externally powered. The source can provide current flows or static potentials. These can be of direct or alternating polarities, continuous or discontinuous, uninterrupted, pulsed, or interrupted, unmodulated, frequency modulated, or amplitude modulated, unvaried, randomly varied, or regularly varied, as possible and suitable. Source modules (168, 170) can be manually, programmatically, or feedback controlled and can incorporate appropriate sensors.

The parallel source modules are designed to operate on unit blocks as proposed in the preferred and alternative embodiments.

Addressable-control source modules (FIG. 8D), (170) may be used with the data and real-time adapter modules (162). The circuitry of each addressable control source module communicates with, controls or is controlled by external controllers through input-output jacks (226), the data and real time adapter module to source module connector (164).

Self-metering or externally-controlled sources can maintain continuous or programmatically defined levels of induced effects based on simultaneous monitoring of the point arrays.

Description—Attachable Accessories

Accessory attachments (FIGS. 7A, 7B, 7C) outwardly resemble one another except for connectors such as wires or tubes attached to some of them and varying thicknesses of the completed assemblies. One or more can be layered between the operational assembly (172, 174, 176) and the visco-elastic pad (178) to provide supplementary stimuli, including electrical or magnetic fields, physical vibration, and heat or cold. Attachment points (148) align the layers.

Grommets (152) reinforce the attachment points.

### DETAILED DESCRIPTION OF THE ALTERNATIVE EMBODIMENT

The alternative [clinical] embodiment differs from the preferred [portable] embodiment in one respect. Each unit block is fitted (FIG. 9A-9D) with a clinical accessory mount designed to securely hold devices such as hypodermic needles, topical applicators, or sensors connected through a tubing or wiring harness to an external device. In the case of hypodermic or topical injectors, the external device may be standard medical drip bags or pumps. Devices such as acupuncture needles would be connected to standard power sources now in common use by practitioners in the field.

Accessory components of the system incorporate openings (FIGS. 7A, 7C) (150) allowing access to the clinical accessory mount on each unit point block of the alternative embodiment. The components are arranged with gaps (FIG. 7C) (186) that permit tubing and wiring harnesses to run unimpeded. Grommets (152) reinforce these openings.

### ADVANTAGES

From the description above, a number of advantages of my therapeutic device become evident:

The novel and unexpected benefits point arrays offer as an enhanced physical stimulus over an area of a subject's body are reflected in the use of flexible, planar arrangements of ferromagnetic and diamagnetic points to enhance electrical current and magnetic fields used in therapy.

Because they are planar and relatively thin, this invention permits application of a variety of stimuli and intensities singly or in arbitrary combinations through additional accessory assemblies.

The flexible and modular design of this invention accommodate needs for portability and simplicity of operation in the field. It accommodates intensive use of invasive procedures, bulky accessories, and complex control systems in clinical settings. Finally, clinical and scientific research is encouraged by easy integration of monitoring systems for a wide range of parameters.

This invention offers treatment modes seen to relate to an unprecedented range of major medical disciplines of our time, including allopathic medicine, Traditional Chinese Medicine, folkloric medicine, herbalism, physical therapy, sports medicine, therapeutic massage, and more.

### OPERATION—Preferred [Portable] Embodiment—FIGS. 6B, 7A, 8A-D

Operation of this invention is typically by application of the active side to a surface of a subject's body. This is commonly achieved by first assembling (FIG. 7A) an operational assembly (172, 174, 176)—including an adapter module (160, 162) and one, two, or no source modules (168, 170)—with optional accessories (180) and the visco-elastic pad attachment (178). This combination is placed on a firm surface such as a massage table. The subject then lies upon or reclines against the active side of the device.

The point arrays and the subject's surface are deformed by their mutual contact so as to exert a relatively uniform pressure over the area of contact. The elasticity of human skin ensures that even very sharp points (100, 104, 110, 112, 114, 116, 122, 124, 130, 132) do not puncture it. Subjects report little or no discomfort from the contact.

An adapter module (160, 162) is required for operation of the device. The electro-mechanical adapter module (160) permits each unit block to be switched to a closed circuit condition permitting electrical potential or current flow from galvanic action induced by the adjacent ferromagnetic and diamagnetic points contacting the subject's skin.

The adapter modules further allow each unit block to be switched to one of two circuits which can be open (no current flow) or closed by an attached parallel source module (168). The source modules may induce electrical currents or potentials of any physical nature possible. They may provide simultaneous metering of electrical conditions between the two sets of connected points in the connected blocks, or they may allow simultaneous metering of those conditions as responsive changes are created in the subject.

The simplest and least expensive electromechanical adapter module (160) has source on-off-option switches (210) with 9 poles and three states. The states set the connected blocks open (no current), closed to themselves (galvanic effects in parallel) or connected to the source module plugged in to its connector (214). More elaborate versions of the electro-mechanical on-off-option source switches (210) accomodate differing combinations of effects and metering through multiple circuits and switch states.

The data and real-time adapter module (162) provides digital (226) and analog (228) interfaces to the unit blocks and controllers built in to the adapters themselves. With these adapters, computer-based, algorythmic or automated controls may be designed and built as needed for therapeutic or research goals. Displays (222), e.g. LED readouts, can optionally provide direct information apart from attached computers or other devices regarding the state[s] of elements within, attached to, or affected by the adapter module.

Addressable control source modules (170) may be used with the data and real-time adapter modules (162). The circuitry of each addressable control source module communicates with, controls, or is controlled by internal components, adapter module controls or external devices. They may generate, monitor, or modulate electromagnetic forces severally and in combination, limited only by the granularity of the connections to the point arrays and circuitry connecting them with those points.

Once the subject and the device have been placed in proper contact, the switch settings on the adapter modules (160, 162) are set in accordance with the effects or monitoring desired.

Setting source selector switches (208) on the electromechanical adapter (160) to the center position allows galvanically-induced micro-currents to flow within each unit block. Setting some number of the source selector switches to one of the source on-off-option switches allows those blocks to be disconnected (open circuit), their galvanically induced micro-currents to flow in parallel as one circuit, or connected to an attached source module. That source module can then provide some induced effect, monitor electromagnetic conditions, or both.

Settings are revised as desired during a course of treatment or observation.

### OPERATION—Alternative [Clinical] Embodiment—FIGS. 7A-B, 9A-D

The single difference between the preferred (portable) and alternative (clinical) embodiments of this invention is the incorporation of a clinical accessory mount (128) in each unit block. This mount securely latches the injector, applicatior, or sensor in place.

The additional step of attaching devices to the clinical accessory mounts (128) precedes assembly of the operational assembly with its attachments (FIG. 7A-B). This present invention allows for their use during the subsequent course of treatment or observation.

### CONCLUSION, RAMIFICATIONS, AND SCOPE

Accordingly, the reader will see that this invention incorporates new and novel technologies for therapy and bioedical research.
First its use of point arrays offers a broad subject area, non-invasive and gentle therapeutic stimulus.
Further, metal points mixing ferromagnetic and diamagnetic materials create galvanic micro current therapeutic stimuli.
Further, mixing ferromagnetic and diamagnetic materials in point arrays greatly potentiates the effectiveness of magnetic field stimuli.
Further the point arrays similarly potentiate applied electromagnetic stimuli in ways not observed by the single point sources employed in other therapeutic devices.
Further, heretofore separate modes of therapy are combined in a practical, expeditious, and unprecedented manner.
Further, the modular and extensible design is adaptable to portable field use and to intensive clinical use. Its extensibility accommodates heretofore unexplored subjects of quantifiable scientific research through sensors and control systems already developed and yet to be developed.

Embodiments not herein illustrated may incorporate more complex and detailed circuitry to extend their functionality, For example, the source modules, adapter modules, and the arrangement of connections between points can each vary from embodiments that affect all points together (in parallel) to the other extreme of separately monitoring and controlling arbitrary groups of individual points severally or together for forces, sensors and controllers of arbitrary degrees of complexity. It is anticipated that this characteristic architecture will permit research and therapies unprecedented in their scope.

Digital technology permits active addressing, control, and monitoring of any arbitrary groups of points, sources and conditions, limited only by the physical contruction of the point arrays and circuitry. The element of change over time, especially changes responsive to a subject's conditions, is another ramification implicit in this present invention's architecture.

An alternative, simpler, embodiment of this invention, not illustrated, is possible in which the alternating point rows in each unit block are directly connected (“shorted”). This embodiment would permit always-on galvanically-induced micro-current flows without controls. A simple adapter module that would mimic this always-on version when used with the proposed embodiments would be a connector plug closing each unit block's circuit at the jack.

Three types of accessory heat sources, not illustrated, envisioned are (a) a convenient electrical pad connected to household power, (b) a liquid-filled bladder with stoppers suitable for chemical reaction-based heating [or cooling], and (c) a pressurized liquid system utilizing a separate heater and pump to circulating temperature-controlled substances through a tubes-and-bladder system.

An electrical heating pad is appropriate to portable uses. Pressurized substances accessory systems are suitable for a clinical setting.

Accessory magnetic field boosters covering localized or wide areas may utilize permanent magnets (FIG. 7B) or electromagnets driven by external power supplies.

The types of arrays of points are themselves open to variation within the scope of this invention. Arrangements of points may be non-planar, nonuniformly sharp or dull, or randomised by material or locations. Points may not be parallel with each other. Points may not hold fixed positions with respect to each other. Points may be moved or impelled separately or in groups.

The materials of the point arrays and suppports will undergo development and variation as research uncovers phenomena of interest that arise from or are responsive to differences in those materials.

Although the description above contains many specificities, these should not be construed as limiting the scope of the invention but as merely providing illustrations of some of the presently preferred embodiments of this invention. For example, the density, sharpness, type of electrical conductors or semiconductors connecting the points, constituent materials and arrangement of the points can assume all physically possible variations, and it is expected that, with further research, more novel and unexpected benefits will be discovered in other combinations of this basic combination of elements.

Thus the scope of the invention should be determined by the appended claims and their legal equivalents, rather than by the examples given.

1. A therapeutic device comprising
a. an array of points, and
b. said array protruding from or upon a supporting material or structure.

2. The device of claim 1 wherein said support is flexible.

3. The device of claim 1 wherein said support is magnetized.

4. The device of claim 1 wherein said points are made with metal.

5. The device of claim 4 wherein said points are of two classes:
diamagnetic and ferromagnetic.

6. The device of claim 5 wherein said classes of points are assembled in some alternating arrangement.

7. The device of claim 6 wherein said points are interconnected with electrical conductors comprising:
a. interconnections within members of a class of points, and
b. switched connections between classes of points.

8. The device of claim 7 wherein said interconnections are further divided into separably operable subdivisions.

9. The device of claim 7 wherein said interconnections are monitored or metered.

10. The device of claim 4 wherein provision is made to apply or permit electrical current or static potential to some, all, or none of the points.

11. The device of claim 10 comprising:
a. a variety of optional power and signal sources,
b. controls for said sources.

12. The device of claim 10 wherein said sources provide:
a. optional modulation or alteration of the forces, and
b. variations or variability in characteristics of induced forces,
c. optional metering, sensing, or measurement of forces extant within the circuitry.

13. The device of claim 1 wherein said device accommodates additional sources of electromagnetic forces, fields, potentials, flows and modulations thereof including, but not restricted to very high and very low frequencies, very high and very low field strengths, continuous and discontinuous in time, repetitive and random variation in characteristics, and manual or automated modulation through sensors, feedback, algorithms or scripts.

14. The device of claim 1 wherein said device accommodates additional sources of heat or cold.

15. The device of claim 14 wherein there is modulation of an optional heat source comprising electrical or heated liquid sources.

16. The device of claim 1 wherein said device exerts even pressure on the subject by means of a pad.

17. The device of claim 1 wherein said device accommodates attachment of a variety of accessories.

18. The device of claim 17 wherein said accessories comprise one or more hypodermic needles and tubing connecting them to a source of injectant, said hypodermic needles so mounted as to remain inserted through a course of treatment.

19. The device of claim 17 wherein said accessories provide means of application of other therapies singly or in combination including, but not limited to, electromagnetic forces, acupuncture needles, vibration, distension and pressure.

20. The device of claim 17 wherein said accessories support monitoring of or themselves monitor therapies, treatment conditions, or patient reactions, and modify their own or other processes accordingly by means including, but not limited to, feedback loops, algorithms, conditional logic, scripts, operator preferences, sensors themselves or via external devices.
